(19) United States
(12) Patent Application Publication
(10) Pub. No.: US 2025/0303630 A1
(43) Pub. Date: Oct. 2, 2025

(54) SYSTEMS AND METHODS FOR PREFORM STERILIZATION

(71) Applicant: NIAGARA BOTTLING, LLC, Diamond Bar, CA (US)

(72) Inventors: Leroy Magwood, Rancho Cucamonga, CA (US); Jingxin Guo, Pomona, CA (US); Melissa Cronin, Alhambra, CA (US); Dagmara Jagoda Czajka, Brea, CA (US)

(21) Appl. No.: 19/092,621

(22) Filed: Mar. 27, 2025

Related U.S. Application Data
(60) Provisional application No. 63/570,676, filed on Mar. 27, 2024.
(60) Provisional application No. 63/570,686, filed on Mar. 27, 2024.
(60) Provisional application No. 63/571,894, filed on Mar. 29, 2024.
(60) Provisional application No. 63/638,796, filed on Apr. 25, 2024.
(60) Provisional application No. 63/687,102, filed on Aug. 26, 2024.
(60) Provisional application No. 63/740,628, filed on Dec. 31, 2024.

(51) Int. Cl. B29C 49/42; A61L 2/18; B08B 6/00; B29K 67/00; B29L 31/00

(52) U.S. Cl. CPC B29C 49/42418; A61L 2/186; B08B 6/00; A61L 2202/23; B29K 2067/003; B29L 2031/7158

(57) ABSTRACT

A method for manufacturing a bottle may including preheating a preform to an internal temperature with an airflow directed within a deduster unit. A method for manufacturing a bottle may also include sterilizing the preheated preform with a hydrogen peroxide solution. A method for manufacturing a bottle may further include forming a bottle from the preheated preform using a blow molding process.

### CROSS-REFERENCE TO RELATED APPLICATIONS

This application claims priority to U.S. Provisional Patent Application No. 63/570,676, filed Mar. 27, 2024, and to U.S. Provisional Patent Application No. 63/570,686, filed Mar. 27, 2024, and to U.S. Provisional Patent Application No. 63/571,894, filed Mar. 29, 2004, and to U.S. Provisional Patent Application No. 63/638,796, filed Apr. 25, 2024, and to U.S. Provisional Patent Application No. 63/687,102, filed Aug. 26, 2024, and to U.S. Provisional Patent Application No. 63/740,628, filed Dec. 31, 2024, the entire contents of all of which are incorporated herein by reference.

### FIELD

The present disclosure relates to bottling lines for liquid containers and, more particularly, to sterilization processes for aseptic bottling lines.

### BACKGROUND

Aseptic bottling lines can be used in the food, beverage, and/or pharmaceutical industries to ensure that products are packaged in a sterile environment. Aseptic bottling lines can extend the shelf life of perishable products without requiring refrigeration or added preservatives by preventing microbial contamination during the packaging process. Thus, in aseptic bottling lines, any packaging materials (such as bottles) may be sterilized to ensure they are free from any contaminants. Hydrogen peroxide (H₂O₂) sterilization processes provide a variety of technical benefits when used in aseptic bottling lines. As a sterilization agent, hydrogen peroxide may be effective against a wide range of microorganisms, including bacteria, viruses, and fungi (such as yeasts and spores). Furthermore, hydrogen peroxide is compatible with a variety of packaging materials, including polymers, metals, and glass. Hydrogen peroxide also acts quickly to sterilize surfaces, allowing aseptic bottling lines that use hydrogen peroxide sterilization processes to maintain a high throughput. After application, hydrogen peroxide decomposes into water and oxygen, both of which are generally harmless to humans and do not affect the packaged product's taste or quality. Additionally, the decomposition products of hydrogen peroxide (e.g., water and oxygen) are environmentally benign. Accordingly, hydrogen peroxide sterilization processes generally do not generate hazardous waste or require complex disposal processes.

### SUMMARY

Systems, apparatuses, methods, and techniques described herein improve aseptic bottling lines that use hydrogen peroxide sterilization processes by pre-heating bottling preforms before they are sterilized with a hydrogen peroxide solution. Pre-heating the preform before hydrogen peroxide sterilization provides a variety of technical benefits to the overall aseptic bottling process. For example, pre-heating the preform increases the surface temperature of the preform. When hydrogen peroxide is applied to a warmer surface, it tends to evaporate more quickly than when it is applied to a cooler surface. This faster evaporation reduces the amount of hydrogen peroxide residue on the surface of the preform after the sterilization process. Furthermore, because hydrogen peroxide decomposes into water and oxygen in the presence of heat, pre-heating the preform accelerates the decomposition, increasing the rate at which hydrogen peroxide is decomposed into its benign components (e.g., water and oxygen), which may increase the throughput of the aseptic bottling line and reduce the migration of hydrogen peroxide downstream. Pre-heating the preform may also reduce the likelihood of hydrogen peroxide vapor condensing on the surface of the preform. Additionally, in cooler ambient conditions, the contrast between the cooler surface of the preform and warmer hydrogen peroxide vapor can lead to condensation forming on the preform surface, resulting in liquid hydrogen peroxide remaining on the preform surface after sterilization.

Additionally, hydrogen peroxide is more reactive at higher temperatures. This increased reactivity enhances its ability to sterilize microorganisms (e.g., by destroying cell walls). For example, the decomposition of hydrogen peroxide into water and oxygen releases free radicals such as hydroxyl radicals. These free radicals may be highly effective in breaking down the cellular components of pathogens. As previously described, hydrogen peroxide decomposes more quickly at higher temperatures. Thus, when hydrogen peroxide is brought into contact with the pre-heated surface of the preform, the condensation of hydrogen peroxide can be avoided, while more active hydrogen peroxide vapor would decompose and release free radicals more quickly, allowing for shorter exposure times to achieve disinfection or sterilization, further allowing for an increased throughput in the aseptic bottling line.

In some aspects, the techniques described herein relate to a method for manufacturing a bottle including: preheating a preform to an internal temperature with an airflow directed within a deduster unit; sterilizing the preheated preform with a hydrogen peroxide solution; and forming a bottle from the preheated preform using a blow molding process.

In some aspects, the techniques described herein relate to a system for manufacturing a bottle including: a deduster unit configured direct an airflow toward a preform to preheat the preform to an internal temperature; a hydrogen peroxide sterilization unit configured to sterilize the preheated preform to an internal temperature; and a blow molding unit configured to form a bottle from the preform.

In some aspects, the techniques described herein relate to a method for manufacturing a bottle including: preheating a preform to an internal temperature with a heating element; sterilizing the preheated preform with a hydrogen peroxide solution; and forming a bottle from the preheated preform using a blow molding process.

In some aspects, the techniques described herein relate to a system for manufacturing a bottle including: a preparation unit configured to preheat a preform to an internal temperature; a hydrogen peroxide sterilization unit configured to sterilize the preheated preform to an internal temperature; and a blow molding unit configured to form a bottle from the preform.

In some aspects, the techniques described herein relate to a sterilization system for a preform, the sterilization system including: a first conduit configured to carry H₂O₂; a second conduit configured to carry air; an evaporator fluidly coupled to the first conduit and the second conduit and configured to vaporize the H₂O₂ and the air, creating vaporized H₂O₂; a nozzle fluidly coupled to the evaporator and configured to deliver the vaporized H₂O₂ to the preform; and a heater configured to heat at least one of the H₂O₂ or the air prior to the at least one of the H₂O₂ or the air entering the evaporator.

In some aspects, the techniques described herein relate to a sterilization system for a preform, the sterilization system including: a first conduit configured to carry H₂O₂; a second conduit configured to carry air; an first cell fluidly coupled to the first conduit and the second conduit and configured to heat the H₂O₂ to within a first temperature range; a second cell fluidly coupled to the first cell and configured to heat the H₂O₂ to within a second temperature range, the second temperature range being greater than the first temperature range; and a nozzle fluidly coupled to the second cell and configured to dispense the H₂O₂ from the second cell toward the preform.

In some aspects, the techniques described herein relate to a sterilization system for a preform, the sterilization system including: an evaporator configured to receive H₂O₂ and heat the H₂O₂, creating vaporized H₂O₂; a nozzle positioned downstream from the evaporator and fluidly coupled to the evaporator, the nozzle being configured to deliver the vaporized H₂O₂ to the preform; and a sensor positioned downstream from the evaporator and configured to detect a temperature of the vaporized H₂O₂ downstream from the evaporator.

In some aspects, the techniques described herein relate to a sterilization system for a preform, the sterilization system including: a sterilizing agent applicator configured to apply a sterilizing agent to an interior of the preform; an oven configured to heat the preform; and a microwave configured to at least partially vaporize the sterilizing agent.

In some aspects, the techniques described herein relate to a method of sterilizing a preform including: applying a sterilizing agent to an interior of the preform; heating the preform and the sterilizing agent in an oven; and directing microwaves toward the preform when the preform is in the oven to apply a secondary heating process to the sterilizing agent.

Other examples, embodiments, features, and aspects will become apparent by consideration of the detailed description and accompanying drawings.

### BRIEF DESCRIPTION OF THE DRAWINGS

FIG. 1 is a schematic illustration of an example aseptic bottling line, according to some embodiments.

FIG. 2 is a schematic illustration of an example preparation unit of FIG. 1, according to some embodiments.

FIG. 3 is a flowchart of an example process for manufacturing a bottle using the bottling line of FIGS. 1 and 2, according to some embodiments.

FIG. 4 is a schematic illustration of an example preparation unit of FIG. 1, according to some embodiments.

FIG. 5 is a flowchart of an example process for manufacturing a bottle using the bottling line of FIGS. 1 and 4, according to some embodiments.

FIG. 5A is a graph depicting the results of a test subjecting preforms at various incoming temperatures to an H₂O₂ sterilization process and then measuring a resultant moisture retention on the preforms.

FIG. 6 is a schematic illustrating portions of a hydrogen peroxide sterilization unit of the bottling line of FIG. 1 for sterilizing the preform of FIG. 1, according to some embodiments.

FIG. 7 is a schematic illustrating portions of a hydrogen peroxide sterilization unit of the bottling line of FIG. 1 for sterilizing the preform of FIG. 1, according to the same or other embodiments.

FIG. 8 is a schematic illustrating portions of a hydrogen peroxide sterilization unit of the bottling line of FIG. 1 for sterilizing the preform of FIG. 1, according to the same or other embodiments.

FIG. 9 is a schematic illustrating a sensor monitoring system of the hydrogen peroxide sterilization unit of FIG. 8.

FIG. 10 is a flow chart illustrating a method for measuring a temperature of a sterilizing fluid.

FIG. 11 is a schematic illustrating a preform sterilization system for use with the bottling line of FIG. 1 according to some embodiments.

FIG. 12 is a schematic illustrating a preform sterilization system for use with the bottling line of FIG. 1 according to another embodiment.

FIG. 13 is a flow chart illustrating a method of sterilizing the preform of FIG. 1.

In the drawings, reference numbers may be reused to identify similar and/or identical elements.

### DETAILED DESCRIPTION

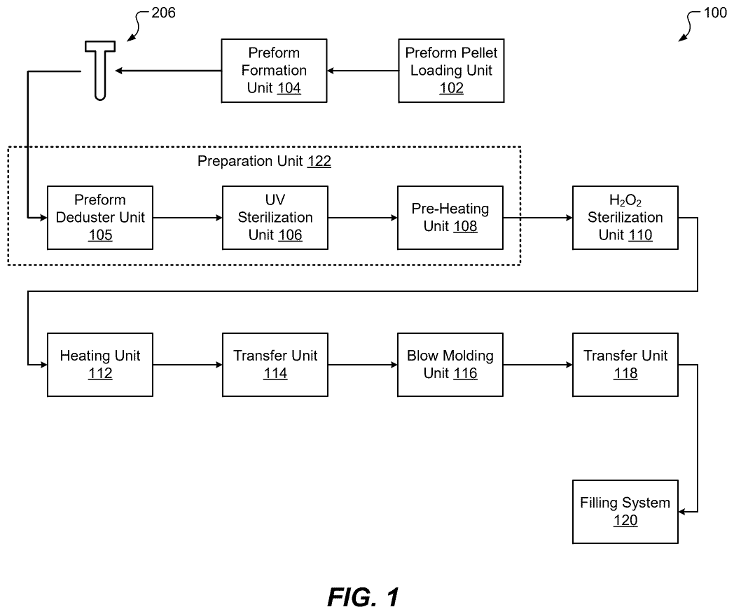

FIG. 1 is a schematic illustration of an aseptic bottling line 100. As illustrated in FIG. 1, some examples of the bottling line 100 include a preform pellet loading unit 102, a preform formation unit 104, a preparation unit 122, a hydrogen peroxide sterilization unit 110, a heating unit 112, a transfer unit 114, a blow molding unit 116, a transfer unit 118, and/or a filling system 120. The preform pellet loading unit 102 may provide a steady and controlled supply of raw material such as polymer pellets to the preform formation unit 104. In various implementations, the pellets may be polyethylene terephthalate (PET) pellets, high-density polyethylene (HDPE) pellets, polypropylene (PP) pellets, low-density polyethylene (LDPE) pellets, polycarbonate (PC) pellets, and/or polylactic acid (PLA) pellets, etc.

In some examples, the preform pellet loading unit 102 is a gravity feed hopper. In various implementations, the preform pellet loading unit 102 is a vacuum loader. In some examples, the preform pellet loading unit 102 is a screw feeder. In various implementations, the preform pellet loading unit 102 is a weigh belt feeder. In some examples, the preform pellet loading unit 102 is a loss-in-weight feeder. In various implementations, the preform pellet loading unit 102 is a pneumatic conveying system. The preform formation unit 104 may receive the pellets from the preform pellet loading unit 102, melt the pellets, and inject the melted polymer pellets into molds that shape a preform 206. After the polymer cools and solidifies into the preform 206 within the mold, the mold may be opened and the preform formation unit 104 may provide the preform 206 to the preparation unit 122. In various implementations, the preform formation unit 104 may form the preform via an extrusion process.

The preparation unit 122 prepares the preform 206 for sterilization treatment by the hydrogen peroxide sterilization unit 110. For example, the preparation unit 122 may remove dust, debris, or other foreign objects from the preform 206, eliminate or inactivate microbes or pathogens, and/or pre-heat the preform 206 to a temperature suitable for hydrogen peroxide (H₂O₂) sterilization. The preparation unit 122 may include a preform deduster unit 105, a UV sterilization unit 106, and/or a pre-heating unit 108.

The preform deduster unit 105 may direct an airflow at the preforms 206 to remove dust or other foreign bodies from the surface of the preforms 206. In some implementations, the preform deduster unit 105 may direct compressed air into an interior of the preform 206. The compressed air flows through the interior of the preform 206 and removes the dust or other foreign bodies, which are carried out of the preform 206 by the compressed air. In other implementations, the preform deduster unit 105 may direct compressed ionized air into the interior of the preform 206. The ionized air may exert an electrostatic attraction toward the dust or other foreign bodies to remove the dust more effectively from the surface of the preform 206. In various implementations, the preform deduster unit 105 uses heated compressed air, or heated compressed ionized air, to pre-heat the preform 206. The preform deduster unit 105 may provide the preform 206 to the UV sterilization unit 106.

The UV sterilization unit 106 may use UV light, such as UV light in the C spectrum (UVC) light to sterilize the surface of the preforms 206. In various implementations, the UV sterilization unit 106 uses UV light to sterilize and/or pre-heat the preform 206. The UV sterilization unit 106 may provide the preform 206 to the pre-heating unit 108.

The pre-heating unit 108 may—for example, in implementations where the UV sterilization unit 106 does not pre-heat the preform 206—pre-heat the preform 206 using one or more heating elements. In implementations where the UV sterilization unit 106 pre-heats the preform 206, the pre-heating unit 108 may further pre-heat the preform 206 or be omitted.

In various implementations, the preform deduster unit 105 and the UV sterilization unit 106 are separate units. In some examples, the preform deduster unit 105 and the UV sterilization unit 106 are a combined unit. In various implementations, the UV sterilization unit 106 and the pre-heating unit 108 are separate units. In some examples, the UV sterilization unit 106 and the pre-heating unit 108 are a combined unit. In various implementations, the pre-heating unit 108 is omitted and the preform deduster unit 105, and optionally also the UV sterilization unit 106, performs the entirety of the pre-heating. In various implementations, the deduster unit 105 pre-heats the preform 206 until its internal temperature is in a range of between about 45° and about 50° Celsius (C). For example, the deduster unit 105 may preheat the preform 206 until its internal temperature reaches about 45° C., 45.5° C., 46° C., 46.5° C., 47° C., 47.5° C., 48° C., 48.5° C., 49° C., 49.5° C., or about 50° C.

The UV sterilization unit 106 or the pre-heating unit 108 may provide the pre-heated preform 206 to the hydrogen peroxide sterilization unit 110. The hydrogen peroxide sterilization unit 110 may receive the pre-heated preform 206 and apply hydrogen peroxide (for example, a hydrogen peroxide solution or vapor) to the pre-heated preform 206 for sterilization. Since the surface of the preform 206 is pre-heated, the hydrogen peroxide may evaporate more quickly or avoid condensation and leave less residue on the surface of the preform 206 while providing active free radicals for sterilization. Thus, less hydrogen peroxide may also migrate downstream from the hydrogen peroxide sterilization unit 110. Additionally, as less exposure time may be required for the hydrogen peroxide to neutralize contaminants, the hydrogen peroxide sterilization unit 110 may be able to sterilize the preforms 206 more quickly, allowing for an improved throughput for the bottling line 100. Ionized air blown onto the preform 206 by the deduster unit 105 can also help the distribution of hydrogen peroxide vapor inside preform 206, which helps the cleaning process. The hydrogen peroxide sterilization unit 110 may provide the sterilized preform 206 to the heating unit 112.

The heating unit 112 may heat the preform 206 to a correct temperature for blow molding, which makes the polymer preform 206 soft and malleable. In various implementations, the heating unit 112 heats the preform 206 to a temperature in a range between about 100° C. and about 120° C. After heating the preform 206, the heating unit 112 may provide the heated preform 206 to the transfer unit 114.

The transfer unit 114 may orient and position the heated preform 206 for the blow molding process. For example, the transfer unit 114 may use mechanical grippers, air jets, and/or conveyers to flip and/or align the heated preforms 206 with a mold of the blow molding unit 116. Accurately positioning the heated preforms 206 ensures that they have the correct orientation and alignment as they are blown into bottles, ensuring the consistency and quality of the final product. The blow molding unit 116 may inject high-pressure air into the preform 206, causing the preform 206 to expand to the shape of the mold, forming the bottle. The transfer unit 118 may transfer the newly formed bottle to the filling system 120, which fills the bottle with the product.

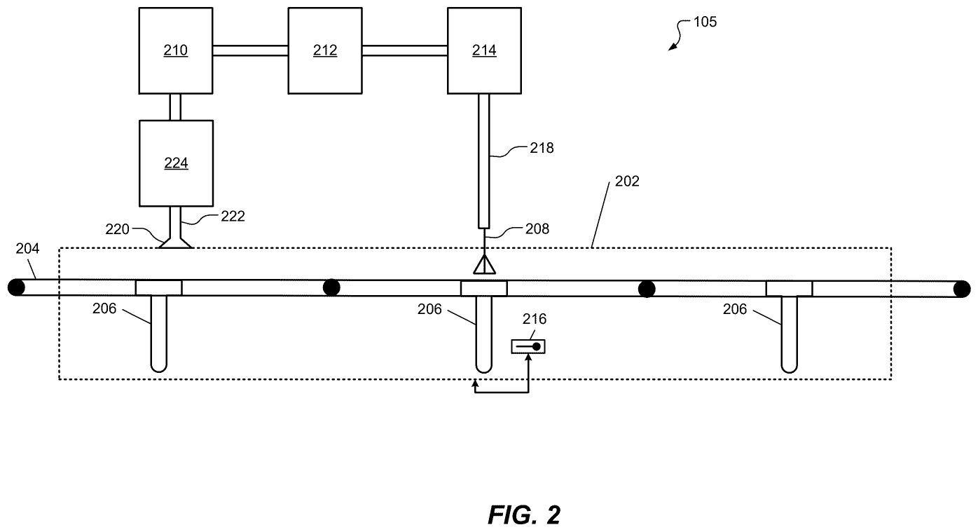

FIG. 2 is a schematic illustration of an example preform deduster unit 105 of FIG. 1. In various implementations, the preform deduster unit 105 includes one or more dedusting zones 202. A conveyer system 204 moves one or more preforms 206 through the dedusting zones 202. Each dedusting zone 202 includes one or more dedusting nozzles 208 to direct compressed air into an interior of the preform 206. In various implementations, multiple dedusting nozzles 208 may direct the compressed air into a single preform 206. One or more dedusting nozzles 208 may also direct the compressed air toward an exterior of the preforms 206.

The preform deduster unit 105 also includes one or more blowers 210, one or more heating elements 212, and one or more ionizers 214. The blower 210 forces the compressed air toward the dedusting nozzles 208. The heating elements 212 pre-heat the compressed air moving toward the dedusting nozzles 208. The ionizer 214 may be optionally included to ionize the compressed air moving toward the dedusting nozzles 208. One or more temperature sensors 216 may be positioned within each dedusting zone 202. In various implementations, the preparation unit 122 monitors temperature signals from the temperature sensors 216 and adjusts the heat output from heating element 212 according to a feedback loop to maintain a setpoint temperature within the dedusting zone 202.

In various implementations, preform deduster unit 105 also includes a supply line 218, an outlet 220, a return line 222, and a filter station 224. The supply line supplies the heated compressed air, which may optionally also be ionized by the ionizer 214, to the one or more dedusting nozzles 208. The dedusting nozzles 208 direct the compressed air toward the surfaces of the preform 206. The air then exits the dedusting zone 202 through the outlet 220 and moves through the return line 222 to the filter station 224. The filter station 224 filters out the dust or other foreign objects which may be moving with the returning air. The filtered air then moves from the filter station 224 back to the blower 210.

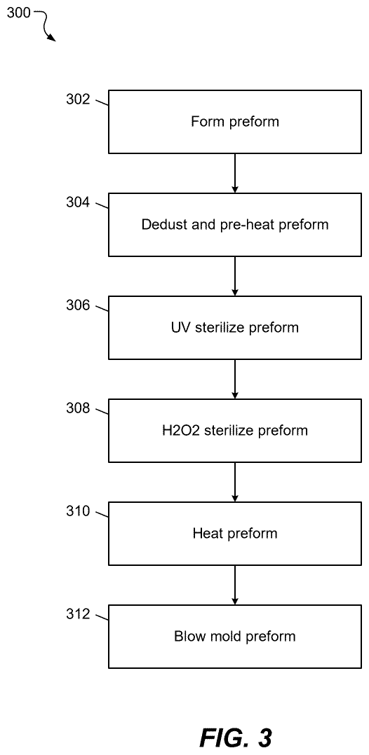

FIG. 3 is a flowchart of an example process 300 for manufacturing a bottle using the bottling line 100 of FIGS. 1 and 2. In the example process 300, the preform pellet loading unit 102 provides pellets to the preform formation unit 104, and the preform formation unit 104 forms a preform 206 (at block 302). In the example process 300, a conveyer system moves the preform 206 from the formation unit 104 to the preform deduster unit 105, which dedusts and pre-heats the preform 206 using heated compressed air (at block 304). The conveyer system then moves the preform 206 to the UV sterilization unit 106, which sterilizes the preform 206 using UV light (at block 306). In the example process 300, the conveyer system moves the preform 206 from the UV sterilization unit 106 to the hydrogen peroxide sterilization unit 110, which sterilizes the preform 206 using hydrogen peroxide (at block 308). In the example process 300, the conveyer system moves the preform 206 from the hydrogen peroxide sterilization unit 110 to the heating unit 112, which heats the preform 206 for blow molding (at block 310). In the example process 300, the conveyer system moves the preform 206 from the heating unit 112 to the transfer unit 114, the transfer unit 114 aligns the preform 206 with a mold of the blow molding unit 116, and the blow molding unit 116 blow molds the preform 206, forming a bottle (at block 312). The transfer unit 118 transfers the final bottle to the filling system 120, where the bottle may be filled with product.

Referring again to FIG. 1, in some implementations, the pre-heating unit 108 is omitted and the UV sterilization unit 106 performs the entirety of the pre-heating. In various implementations, the preform deduster unit 105 does not pre-heat the preform 206. In various implementations, the UV sterilization unit 106 and/or the pre-heating unit 108 pre-heats the preform until its internal temperature is in a range of between about 45° and about 50° Celsius (C). For example, the UV sterilization unit 106 and/or the pre-heating unit 108 may preheat the preform until its internal temperature reaches about 45° C., 45.5° C., 46° C., 46.5° C., 47° C., 47.5° C., 48° C., 48.5° C., 49° C., 49.5° C., or about 50° C.

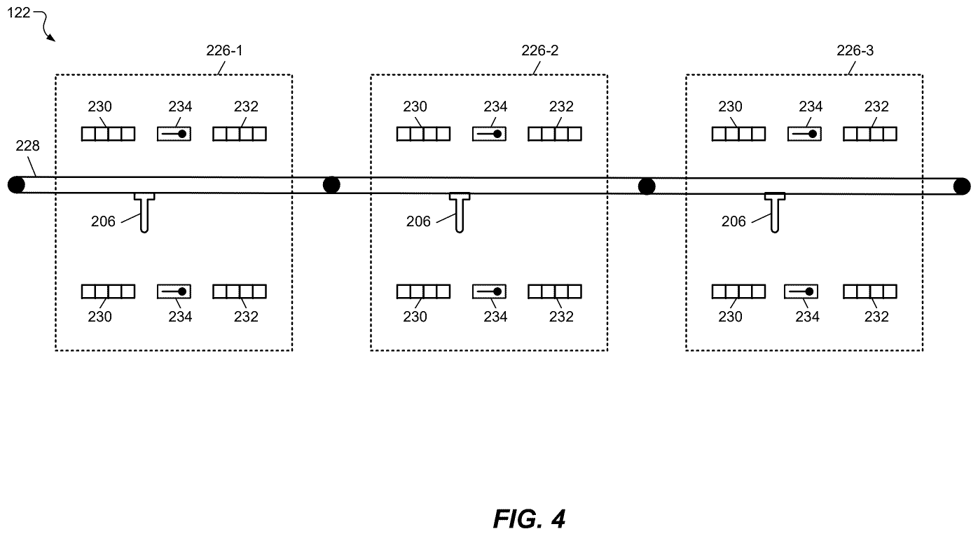

FIG. 4 is a schematic illustration of an example preparation unit 122 of FIG. 1. In various implementations, the preparation unit 122 includes one or more pre-heating zones 226, such as pre-heating zone 226-1, pre-heating zone 226-2, and pre-heating zone 226-3. While three pre-heating zones 226 are shown in the example of FIG. 4, the preparation unit 122 may have any number of pre-heating zones 226, including only one. A conveyer system 228 moves one or more preforms 206 through the pre-heating zones 226, which heat the preforms 206 to the desired internal temperature. In various implementations, each pre-heating zone 226 may be heated to a different temperature to ensure a gradual heat transfer to the preforms 206. One or more heating elements 230 and/or heating elements 232 may be positioned within each pre-heating zone 226. One or more temperature sensors 234 may be positioned within each pre-heating zone 226. In various implementations, the preparation unit 122 monitors temperature signals from the temperature sensors 234 and adjusts the heat output from heating elements 230 and/or heating elements 232 according to a feedback loop to maintain a setpoint temperature within the pre-heating zone 226.

In various implementations, the heating elements 230 comprise UV lamps. In some examples, the heating elements 232 include infrared heaters, quartz tube heaters, ceramic heaters, hot air ovens, induction heaters, and/or halogen lamps. In some examples, the heating elements 230 and/or 232 are evenly distributed within each pre-heating zone 226. In various implementations, the conveyer system 228 rotates the preforms 206 as they move through the pre-heating zones 226. In some examples, the heating elements 230 are omitted. In various implementations, the heating elements 232 are omitted.

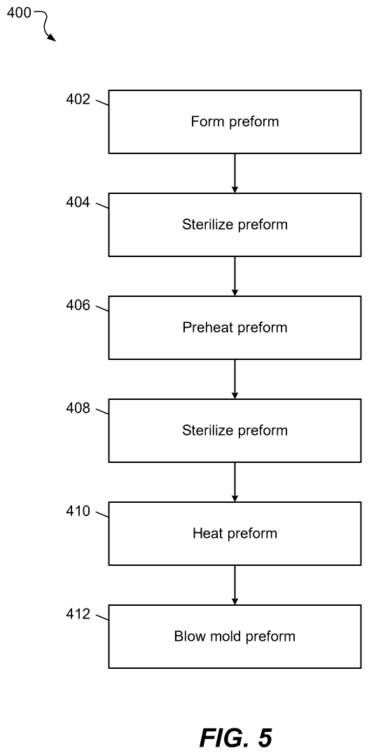

FIG. 5 is a flowchart of an example process 400 for manufacturing a bottle using the bottling line 100 of FIGS. 1 and 4. In the example process 400, the preform pellet loading unit 102 provides pellets to the preform formation unit 104, and the preform formation unit 104 forms a preform (at block 402). In the example process 400, a conveyer system moves the preform from the formation unit 104 to the UV sterilization unit 106, which sterilizes the preform using UV light (at block 404). In the example process 400, the conveyer system moves the preform from the UV sterilization unit 106 to the pre-heating unit 108, which pre-heats the preform (at block 406). In the example process 300, the conveyer system moves the preform from the pre-heating unit 108 to the hydrogen peroxide sterilization unit 110, which sterilizes the preform using hydrogen peroxide (at block 408). In the example process 400, the conveyer system moves the preform from the hydrogen peroxide sterilization unit 110 to the heating unit 112, which heats the preform for blow molding (at block 410). In the example process 400, the conveyer system moves the preform from the heating unit 112 to the transfer unit 114, the transfer unit 114 aligns the preform with a mold of the blow molding unit 116, and the blow molding unit 116 blow molds the preform, forming a bottle (at block 412). The transfer unit 118 transfers the final bottle to the filling system 120, where the bottle may be filled with product.

FIG. 5A is a graph depicting the results of a test subjecting preforms at various incoming temperatures to an H₂O₂ sterilization process and then measuring a resultant moisture retention on the preforms. A temperature difference AT between a temperature of an H₂O₂ flow (e.g., 108 degrees Celsius) and an incoming temperature of the preform was controlled during the test. The preforms, at their various temperatures yielding the plotted AT values, were subjected to the H₂O₂ flow through a test apparatus at both low and high speeds, and the resultant moisture retention on the preforms was subsequently measured. As can be seen from the graph of FIG. 5A, a correlation of greater temperature differences resulting in higher moisture retention was observed. Thus, the pre-heating methods and apparatus described herein in connection with FIGS. 1-5, including the preform deduster unit 105, the UV sterilization unit 106, and/or the pre-heating unit 108, beneficially reduce moisture retention on the preforms 206 by reducing the temperature difference AT between the preform 206 and the H₂O₂ applied at the hydrogen peroxide sterilization unit 110.

The present disclosure provides additional systems and methods improving upon a sterilization process by which a sterilizing agent, such as hydrogen peroxide (H₂O₂), is applied to a preform. The H₂O₂ is vaporized prior to being applied to the preform by combining H₂O₂ and air and evaporating the combination. By vaporizing the H₂O₂, condensation and residual H₂O₂ within the preform is minimized. To further minimize the condensation and residual H₂O₂ within the preform, the preform is heated after application of the vaporized H₂O₂. However, several factors can affect the sterilization process and lead to inadequate sterilization. Particularly, the H₂O₂ may not be completely vaporized prior to applying the H₂O₂ to the preform. Incomplete vaporization may be due to a temperature of the H₂O₂ and/or the air prior to evaporating the combination. Additionally, incomplete vaporization may occur when the combination cools after being evaporated. Particularly, the combination may cool when awaiting application onto the preform.

The present disclosure, therefore, provides a system that improves vaporization of the sterilizing agent prior to applying the sterilizing agent to the preform during the sterilization process. More particularly, the present disclosure includes an additional heating component to heat the H₂O₂ and/or the air to improve vaporization of the H₂O₂ combination. In some embodiments, the present disclosure includes an additional insulation component to maintain a temperature of the H₂O₂ and/or the air in order to properly vaporize the H₂O₂ combination. In some embodiments, the present disclosure includes an additional cooling component to cool the air in order to properly vaporize the H₂O₂ combination.

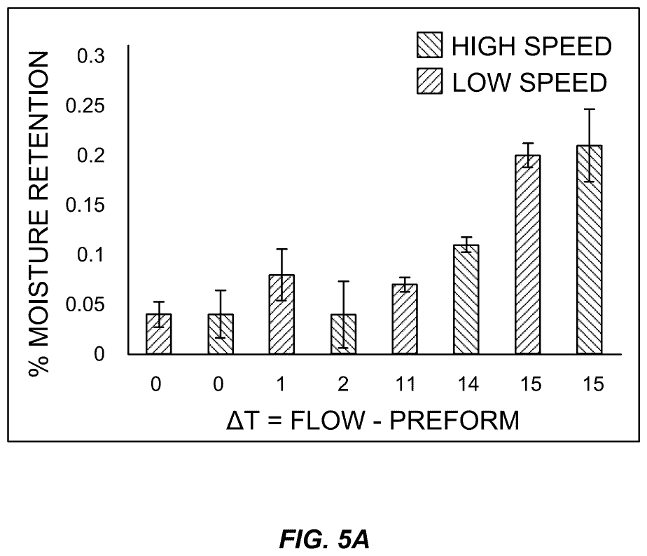

Referring to FIGS. 1 and 6, the hydrogen peroxide sterilization unit 110, or sterilization system 110, includes a vaporized hydrogen peroxide unit, or VHP unit 134. The VHP unit 134 is operable to apply vaporized H₂O₂ to an interior surface of the preform 206. The heating unit 112, or heater 112, heats the preform 206 which further vaporizes any condensed or residual H₂O₂ prior to the preform 206 moving to the blow molding unit 116 (or blow molding station 116). By heating the preform 206 after the vaporized H₂O₂ is applied to the preform 206, the bottling line 100 ensures that the preform 206 is sterilized and prevents condensation of H₂O₂ on the preform leading up to blow molding.

With reference to FIG. 6, the VHP unit 134 includes an H₂O₂ chamber 146, an air chamber 150, an evaporator 154, and a cell 156. The H₂O₂ chamber 146 is fluidly coupled to the evaporator 154 via an H₂O₂ conduit 158. The H₂O₂ conduit 158 transports H₂O₂ from the H₂O₂ chamber 146 to the evaporator 154. Similarly, the air chamber 150 is fluidly coupled to the evaporator 154 via an air conduit 162. The air conduit 162 transports air from the air chamber 150 to the evaporator 154. In other embodiments, the air conduit 162 may be coupled to external air, rather than the air chamber 150 such that the air conduit 162 draws in ambient air. In some embodiments, the H₂O₂ conduit 158 and the air conduit 162 are formed of the same material. In other embodiments, the H₂O₂ conduit 158 and the air conduit 162 may be formed of different materials. The H₂O₂ conduit 158 and/or the air conduit 162 may be formed of HDPE or stainless steel. In other embodiments, the H₂O₂ conduit 158 and the air conduit 162 may be formed of alternative materials.

The evaporator 154 is configured to receive the H₂O₂ and the air, which will be referred to as the combination, and mix and vaporize the combination. The evaporator 154 heats the combination to a vaporization temperature of 108 degrees Celsius. In other embodiments, the vaporization temperature may be below 108 degrees Celsius. In other embodiments, the vaporization temperature may be above 108 degrees Celsius. Once the evaporator 154 heats the combination, the H₂O₂ is vaporized, creating vaporized H₂O₂. The evaporator 154 is fluidly coupled to the cell 156 via an exit conduit 163. The exit conduit 163 may be formed of HDPE or stainless steel. In other embodiments, the exit conduit 163 may be formed of alternative materials. The combination including the vaporized H₂O₂ and the air travels through the exit conduit 163 and into the cell 156. The combination including the vaporized H₂O₂ and the air remains in the cell 156 until the combination is sprayed onto the preform 206 via a nozzle 164. The nozzle 164 is fluidly coupled to the cell 156. For example, the combination sprayed out from the nozzle 164 can form a cloud of vaporized H₂O₂ and air that interacts with surfaces of the preform 206 to sterilize the surfaces.

Under at least some conditions, the evaporator 154 may be insufficient to completely vaporize the H₂O₂, which can cause residual H₂O₂ to remain in the evaporator 154. In some cases, the insufficient vaporization may be caused by a temperature differential between a temperature of the H₂O₂ entering the evaporator 154 and the vaporization temperature. In other words, if the H₂O₂ enters the evaporator 154 at a temperature that is lower than the vaporization temperature, the evaporator 154 may insufficiently vaporize the H₂O₂. To improve the vaporization of the H₂O₂, the H₂O₂ is brought to within a predetermined temperature range (via pre-heating or pre-cooling) prior to entering the evaporator 154.

In some embodiments, the VHP unit 134 may include an H₂O₂ chamber heater 166 configured to heat the H₂O₂ held within the H₂O₂ chamber 146. More particularly, the H₂O₂ chamber heater 166 may be temperature controlled. The H₂O₂ chamber 146 may be set at the predetermined temperature range. When a sensor measures that a temperature of the H₂O₂ within the H₂O₂ chamber 146 falls below the predetermined temperature range, the H₂O₂ chamber heater 166 activates to heat the H₂O₂ within the H₂O₂ chamber 146. The H₂O₂ chamber heater 166 may be an inductive heater, an infrared heater, or a similar heater.

In some embodiments, the VHP unit 134 may include an H₂O₂ conduit heater 168 configured to heat the H₂O₂ conduit 158. For example, the H₂O₂ conduit heater 168 may include an induction heater disposed adjacent the H₂O₂ conduit 158. Since the H₂O₂ conduit 158 is formed of metal, the induction heater inductively heats the H₂O₂ conduit 158. In other embodiments, the H₂O₂ conduit heater 168 may include an infrared heater disposed adjacent the H₂O₂ conduit 158 in order to heat the H₂O₂ conduit 158. In other embodiments, the H₂O₂ conduit heater 168 may include an alternative heater. The H₂O₂ conduit heater 168 may be temperature controlled. More specifically, the H₂O₂ conduit 158 may be set at a predetermined temperature range. When a sensor measures that a temperature of the H₂O₂ within the H₂O₂ conduit 158 falls below the predetermined temperature range, the H₂O₂ conduit heater 168 may heat the H₂O₂ conduit 158. In other embodiments, the H₂O₂ conduit heater 168 may be turned on manually. By heating the H₂O₂ conduit 158 the temperature of the H₂O₂ within the H₂O₂ conduit 158 is increased or maintained. For example, the H₂O₂ naturally tends to experience heat loss causing a decrease in temperature as the H₂O₂ travels through the H₂O₂ conduit 158. By heating the H₂O₂ conduit 158, the H₂O₂ is maintained within or brought to within the predetermined temperature range as the H₂O₂ travels through the H₂O₂ conduit 158.

In some embodiments, the VHP unit 134 may include an H₂O₂ conduit insulator 170 configured to thermally insulate the H₂O₂ conduit 158. The H₂O₂ conduit insulator 170 may include thermal insulation disposed around an outer surface of the H₂O₂ conduit 158. The thermal insulation may be cellulose, polyurethane foam, or a similar insulating material. The H₂O₂ conduit insulator 170 prevents the H₂O₂ from cooling as the H₂O₂ travels through the H₂O₂ conduit 158. As previously mentioned, the H₂O₂ generally decreases in temperature as the H₂O₂ travels through the H₂O₂ conduit 158. By insulating the H₂O₂ conduit 158, the H₂O₂ is prevented from cooling. In some embodiments, the VHP unit 134 may include the H₂O₂ chamber heater 166, the H₂O₂ conduit heater 168, and the H₂O₂ conduit insulator 170. In other embodiments, the VHP unit 134 may include just one of the H₂O₂ chamber heater 166, the H₂O₂ conduit heater 168, and the H₂O₂ conduit insulator 170, or just two of these features in combination.

Similarly, the insufficient vaporization may be caused by a temperature differential between a temperature of the air entering the evaporator 154 and the vaporization temperature. In other words, if the air enters the evaporator 154 at a temperature that is lower than the vaporization temperature, the evaporator 154 may insufficiently vaporize the H₂O₂ within the combination. To improve the vaporization of the H₂O₂, the air may be heated prior to entering the evaporator 154.

In some embodiments, the VHP unit 134 may include an air chamber heater 172 configured to heat the air chamber 150. More particularly, the air chamber heater 172 may be temperature controlled. The air chamber 150 may be set at a predetermined temperature range. When a sensor measures that a temperature within the air chamber 150 falls below the predetermined temperature range, the air chamber heater 172 may heat the air chamber 150. The air chamber heater 172 may be an inductive heater, an infrared heater, or a similar heater.

In some embodiments, the VHP unit 134 may include an air conduit heater 174 configured to heat the air conduit 162. For example, the air conduit heater 174 may include an induction heater disposed adjacent the air conduit 162. Since the air conduit 162 is formed of metal, the induction heater inductively heats the air conduit 162. In other embodiments, the air conduit heater 174 may include an infrared heater disposed adjacent the air conduit 162 in order to heat the air conduit 162. In other embodiments, the air conduit heater 174 may include an alternative heater. The air conduit heater 174 may be temperature controlled. More specifically, the air conduit 162 may be set at a predetermined temperature range. When a sensor measures that a temperature within the air conduit 162 falls below the predetermined temperature range, the air conduit heater 174 may heat the air conduit 162. In other embodiments, the air conduit heater 174 may be turned on manually. By heating the air conduit 162 the temperature of the air within the air conduit 162 is increased or maintained. For example, the air naturally tends to experience heat loss causing a decrease in temperature as the air travels through the air conduit 162. By heating the air conduit 162, the is maintained within or brought to within the predetermined temperature range as the air travels through the air conduit 162.

In some embodiments, the VHP unit 134 may include an air conduit insulator 176 configured to thermally insulate the air conduit 162. The air conduit insulator 176 may include thermal insulation disposed around an outer surface of the air conduit 162. The air conduit insulator 176 may be cellulose, polyurethane foam, or a similar insulating material. The air conduit insulator 176 prevents the air from cooling as the air travels through the air conduit 162. As previously mentioned, the air generally decreases in temperature as the air travels through the air conduit 162. By insulating the air conduit 162, the air is prevented from cooling. In some embodiments, the VHP unit 134 may include the air chamber heater 172, the air conduit heater 174, and the air conduit insulator 176. In other embodiments, the VHP unit 134 may include just one of or any combination of the air chamber heater 172, the air conduit heater 174, and the air conduit insulator 176.

In some embodiments, the air may be cooled prior to entering the evaporator 154. More specifically, when the air is at an increased temperature due to a temperature of the environment, the air may require cooling to optimize the vaporization of the combination. The increased temperature of the air may cause the air to condense, which negatively affect the vaporization of the combination. In some embodiments, a cooling device 178 may be used to cool the air conduit 162. The cooling device 178 may be an air conditioning unit positioned proximate the air conduit 162, or a similar device. The cooling device 178 may be temperature controlled. More specifically, the air conduit 162 may be set at a predetermined temperature range. When a sensor measures that a temperature within the air conduit 162 falls below the predetermined temperature range, the cooling device 178 may cool the air conduit 162. In other embodiments, the cooling device 178 may be turned on manually. In some embodiments, a dryer may be used to dry the air in the air conduit 162. The dryer may be in communication with the air conduit 162 to dry the air in the air conduit 162 when the air has an increased moisture content. The dryer may be integral with the cooling device 178. In other embodiments, the VHP unit 134 may include a separate dryer and a separate cooling device 178. In other embodiments, the VHP unit 134 may solely include either the dryer or the cooling device 178. In other embodiments, the VHP unit 134 may not include the dryer or the cooling device 178.

Additionally, while the evaporator 154 may sufficiently vaporize the H₂O₂, the vaporized H₂O₂ may condense as the vaporized H₂O₂ exits the evaporator 154. Specifically, cooling of the vaporized H₂O₂ causes the vaporized H₂O₂ to condense, leaving residual H₂O₂ in the VHP unit 134. To prevent the vaporized H₂O₂ from cooling, the VHP unit 134 may include an exit conduit heater 180 configured to heat the exit conduit 163. For example, the exit conduit heater 180 may include an induction heater disposed adjacent the exit conduit 163. Since the exit conduit 163 is formed of metal, the induction heater inductively heats the exit conduit 163. In other embodiments, the exit conduit heater 180 may include an infrared heater disposed adjacent the exit conduit 163 in order to heat the exit conduit 163. In other embodiments, an alternative heater may be used to heat the exit conduit 163. The exit conduit heater 180 may be temperature controlled. More specifically, the exit conduit 163 may be set at a predetermined temperature range. When a sensor measures that a temperature within the exit conduit 163 falls below the predetermined temperature range, the exit conduit heater 180 may heat the exit conduit 163. In some embodiments, the exit conduit heater 180 may heat the exit conduit 163 when the vaporized H₂O₂ falls below the vaporization temperature. In other embodiments, the exit conduit heater 180 may be turned on manually. By heating the exit conduit 163, the temperature of the combination within the exit conduit 163 is maintained. For example, the combination generally decreases in temperature as the combination travels through the exit conduit 163. By heating the exit conduit 163, the combination does not cool as the combination travels through the exit conduit 163. Therefore, the combination remains vaporized and condensation of H₂O₂ is prevented.

In some embodiments, the VHP unit 134 may include an exit conduit insulator 182 configured to thermally insulate the exit conduit 163. The exit conduit insulator 182 may include thermal insulation disposed around an outer surface of the exit conduit 163. The thermal insulation may be cellulose, polyurethane foam, or a similar insulating material. The thermal insulation prevents the combination from cooling as the combination travels through the exit conduit 163. As previously mentioned, the combination may tend to decrease in temperature as the combination travels through the exit conduit 163. By insulating the exit conduit 163, cooling of the combination is prevented. In some embodiments, the VHP unit 134 may include the exit conduit heater 180 and the exit conduit 163 insulator. In other embodiments, the VHP unit 134 may include one of the exit conduit heater 180 or the exit conduit insulator 182.

The present disclosure also provides a preform sterilization system that improves vaporization of the sterilizing agent prior to applying the sterilizing agent to the preform during the sterilization process. More particularly, the present disclosure includes a multi-step heating process to precisely control an activation level of the H₂O₂. The multi-step heating process improves upon conventional, single-stage heating and vaporization processes, which can suffer from incomplete vaporization of the H₂O₂ and poorly controlled activation of the H₂O₂, causing condensation and residual H₂O₂ to be left in the system.

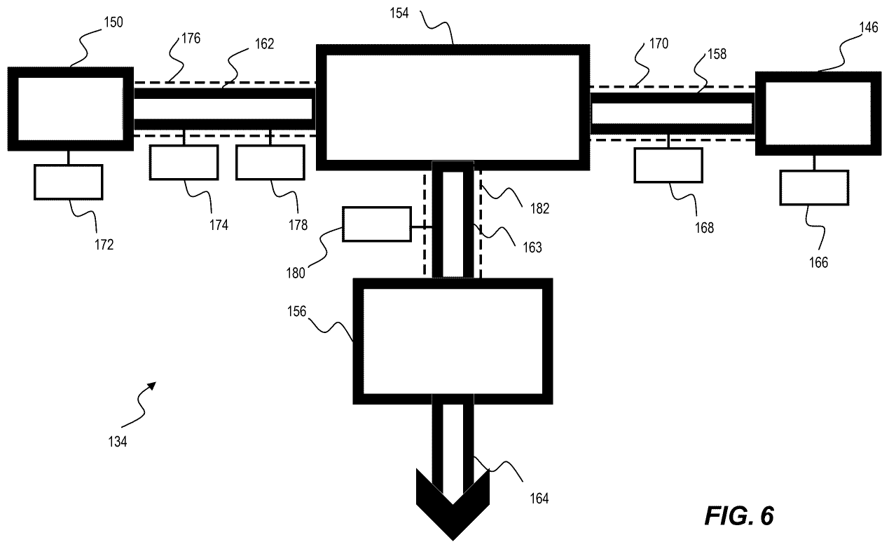

FIG. 7 illustrates an alternative vaporized hydrogen peroxide unit, or VHP unit 534, of the preform sterilization unit 110 (FIG. 1). Features of the VHP unit 534 may be combined with the features of the VHP unit 134 described herein or implemented independently. The VHP unit 534 is operable to apply vaporized H₂O₂ to an interior surface of the preform 206 (FIG. 1).

The VHP unit 534 includes an H₂O₂ chamber 546, an air chamber 550, a first cell 554, a second cell 556, and a nozzle 564. The H₂O₂ chamber 546 is fluidly coupled to the first cell 554 via an H₂O₂ conduit 558. The H₂O₂ conduit 558 transports H₂O₂ from the H₂O₂chamber 546 to the first cell 554. Similarly, the air chamber 550 is fluidly coupled to the first cell 554 via an air conduit 562. The air conduit 562 transports air from the air chamber 550 to the first cell 554. In other embodiments, the air conduit 562 may be coupled to external air, rather than the air chamber 550 such that the air conduit 562 draws in ambient air. In some embodiments, the H₂O₂ conduit 558 and the air conduit 562 are formed of the same material. In other embodiments, the H₂O₂ conduit 558 and the air conduit 562 may be formed of different materials. The H₂O₂ conduit 558 and/or the air conduit 562 may be formed of HDPE or stainless steel. In other embodiments, the H₂O₂ conduit 558 and the air conduit 562 may be formed of alternative materials. The H₂O₂ conduit 558 and the air conduit 562 are coupled to a bottom portion of the first cell 554. Therefore, when the H₂O₂ and the air enters the first cell 554, the H₂O₂ and the air enter into the bottom portion of the first cell 554.

The first cell 554 is configured to receive the H₂O₂ and the air, which will be referred to as the combination, and mix and heat the combination. In some embodiments, the first cell 554 heats the combination to within a first temperature range. When the combination is within the first temperature range, the combination is not yet sufficiently heated for the H₂O₂ to reach a desired activation temperature upon being dispensed from the nozzle 564 toward the preform 206. Thus, the combination within the first cell 554 must still be further heated to reach a second temperature range corresponding to the H₂O₂ reaching the activation temperature upon being dispensed from the nozzle 564. In other embodiments, the first cell 554 may heat the combination to within the second temperature range such that the combination is ready to be dispensed through the nozzle 564. In such embodiments, the second cell 556 may further heat the combination only to maintain the combination within the second temperature range prior to being dispensed from the nozzle 564, as will be discussed further herein.

The first cell 554 is fluidly coupled to the second cell 556 via an exit conduit 563. The exit conduit 563 may be formed of HDPE or stainless steel. In other embodiments, the exit conduit 563 may be formed of alternative materials. The exit conduit 563 is coupled to a top portion of the first cell 554 and a bottom portion of the second cell 556. The combination within the first cell 554 may include some H₂O₂ in a gaseous phase and some H₂O₂ in a liquid phase. The denser liquid H₂O₂ within the first cell 554 tends to accumulate toward a bottom portion of the first cell 554 (i.e., with respect to a gravity vector directed toward the ground). Since the exit conduit 563 is positioned at the top portion of the first cell 554, solely gaseous H₂O₂ exits through the exit conduit 563. In other words, since H₂O₂ in the gas phase is lighter than H₂O₂ in a liquid phase, the gaseous H₂O₂ moves to the top portion of the first cell 554 while liquid H₂O₂ remains in the bottom portion of the first cell 554. Air enters the first cell 554 via the air conduit 562 to push the gaseous H₂O₂ into and through the exit conduit 563. The air is pushed into the first cell 554 at a first flow rate. The first flow rate corresponds with a desired temperature of the gaseous H₂O₂. For example, if a user desires the gaseous H₂O₂ to reach the second temperature range in the first cell 554, the first flow rate may be slower, allowing the gaseous H₂O₂ to spend more time in the first cell 554. Spending additional time in the first cell 554 further heats the gaseous H₂O₂. As another example, if the user desires the gaseous H₂O₂ only to reach the first temperature range in the first cell 554, the first flow rate may be faster such that the gaseous H₂O₂ spends less time in the first cell 554 once the H₂O₂ is in the gas phase. The user may change the flow rate as desired to change the activation of the gaseous H₂O₂ within the first cell 554.

The gaseous H₂O₂ travels through the exit conduit 563 and into the bottom portion of the second cell 556. The gaseous H₂O₂ remains in the second cell 556 until the gaseous H₂O₂ is sprayed onto the preform 206 via the nozzle 564. The nozzle 564 is fluidly coupled to a top portion of the second cell 556. Therefore, solely gaseous H₂O₂ exits the second cell 556 and is sprayed through the nozzle 564 toward the preform 206. In other words, since H₂O₂ in the gas phase is lighter than H₂O₂ in the liquid phase, the gaseous H₂O₂ moves to the top portion of the second cell 556 while the liquid H₂O₂ remains in the bottom portion of the second cell 556. This prevent the liquid H₂O₂ from entering the preform. Since liquid H₂O₂ is prevented from entering the preform 206, residual H₂O₂ within the preform 206 is also prevented. The second cell 556 heats the gaseous H₂O₂ to within the second temperature range. For example, if the first cell 554 solely heats the gaseous H₂O₂ to within the first temperature range, the second cell 556 heats the gaseous H₂O₂ from the first temperature range up to within the second temperature range. As another example, if the first cell 554 heats the gaseous H₂O₂ to within the second temperature range, the second cell 556 further heats the gaseous H₂O₂ only so much as to maintain the gaseous H₂O₂ within the second temperature range.

The second cell 556 additionally includes a second air conduit 568 fluidly coupled to the bottom portion of the second cell 556. The second air conduit 568 pushes air into the second cell 556 at a second flow rate. The air moves the gaseous H₂O₂ through the second cell 556, and into the nozzle at the second flow rate. The second flow rate corresponds to the desired temperature the gaseous H₂O₂ exiting the second cell 556. For example, if the temperature of the gaseous H₂O₂ entering the second cell 556 is close to the desired temperature (i.e., close to or within the second temperature range), the flow rate will be faster, allowing the gaseous H₂O₂ to spend less time heating within the second cell 556. As another example, if the gaseous H₂O₂ entering the second cell 556 is at a relatively lower temperature (i.e., within the first temperature range), the flow rate will be slower, allowing the gaseous H₂O₂ to spend more time heating within the second cell 556. In additional embodiments, the VHP unit 534 may include additional cells to further heat the H₂O₂.

The gaseous H₂O₂ is pushed at the first flow rate in the first cell 554 and the gaseous H₂O₂ is pushed at the second flow in the second cell 556. In some embodiments, the first flow rate is slower than the second flow rate. Therefore, the gaseous H₂O₂ moves slower through the first cell 554 than the gaseous H₂O₂ moves through the second cell 556. This allows the gaseous H₂O₂ to receive more heating in the first cell 554 than in the second cell 556. Specifically, the first cell 554 acts as a primary heater to heat the H₂O₂ to within the first temperature range. Once the gaseous H₂O₂ is in the second cell 556, the gaseous H₂O₂ requires minimal heating to reach to within the second temperature range corresponding to the desired activation temperature of the H₂O₂ exiting the nozzle 564. Since the gaseous H₂O₂ requires minimal heating in the second cell 556, the second cell 556 acts as a more precise heater. That is, a first temperature difference ΔT1 between the temperature of the H₂O₂ within the H₂O₂ chamber 546 and the first temperature range is greater than a second temperature difference ΔT2 between the first temperature range and the second temperature range (i.e., ΔT1>ΔT2). Thus, the H₂O₂ moving through the VHP unit 534 undergoes relatively more heating within the first cell 554 and relatively less heating within the second cell 556. Because lower temperature differences during heating processes tend to correspond with greater accuracy and more controllability, the second cell 556 can heat the gaseous H₂O₂ more precisely to within the second temperature range. Since the H₂O₂ is not heated from its initial temperature to within the second temperature range all at once, but is rather broken into two heating stages within the two cells 554, 556, the heating is more controlled. By controlling the heating of the H₂O₂, condensation within the first and second cells 554, 556 is reduced.

In operation, to sterilize the preform 206, the H₂O₂ enters the first cell 554 via the H₂O₂ conduit from the H₂O₂ chamber 546. Additionally, the air enters the first cell 554 via the air conduit 562 from the air chamber 550. The first cell 554 heats the combination to within the first temperature range. Simultaneously, the air moves into the first cell 554 at the first flow rate. The air moves the H₂O₂ through the first cell 554 and into the exit conduit 563 at the first flow rate. Once the H₂O₂ exits the first cell 554, the H₂O₂ is within the first temperature range. The H₂O₂ enters the second cell 556 from the exit conduit 563 still within the first temperature range. The second cell 556 heats the H₂O₂ to within the second temperature range corresponding to a desired activation temperature of the H₂O₂ upon exiting the nozzle 564. Simultaneously, air moves into the second cell 556 via the second air conduit 568 at the second flow rate. The air pushes the H₂O₂ through the second cell 556 and into the nozzle 564. Once the H₂O₂ enters the nozzle 564, the H₂O₂ is within the second temperature range. The combination including the H₂O₂ and the air is dispersed onto the preform 206 via the nozzle 564 to form a cloud within which the H₂O₂ is at the desired activation temperature. The preform 206 continues moving through the bottling line 100.

The present disclosure also provides a preform sterilization system that improves vaporization of the sterilizing agent prior to applying the sterilizing agent to the preform during the sterilization process. Specifically, in some embodiments, the preform sterilization system includes a sensor positioned in a nozzle for measuring a temperature of the vaporized H₂O₂ while the vaporized H₂O₂ is in the nozzle. Based on a reading received by the sensor, a controller alters the system to maintain the vaporized H₂O₂ at the vaporization temperature.

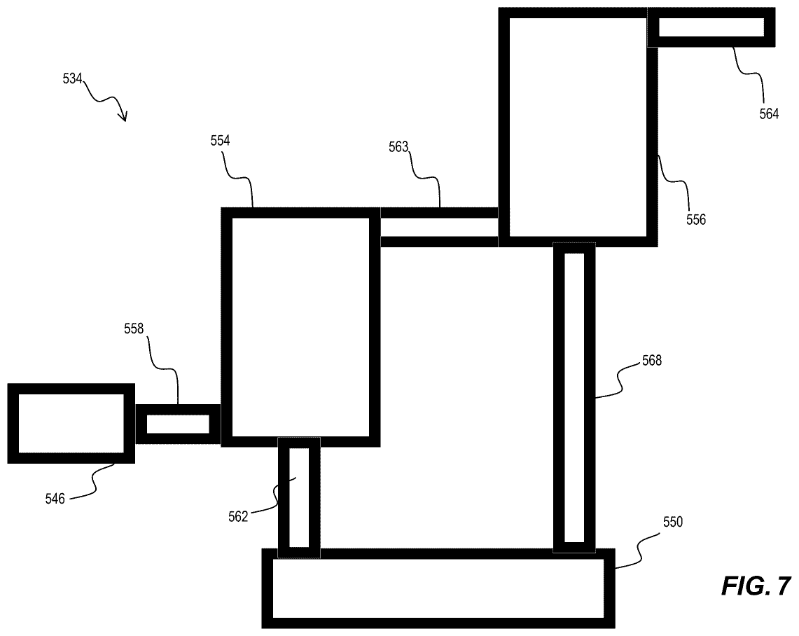

FIG. 8 illustrates an alternative vaporized hydrogen peroxide unit, or VHP unit 634, of the preform sterilization unit 110 (FIG. 1). Features of the VHP unit 634 may be combined with the features of the VHP unit 534 and/or the VHP unit 134 described herein or implemented independently. The VHP unit 634 is operable to apply vaporized H₂O₂ to an interior surface of the preform 206 (FIG. 1).

The VHP unit 634 includes an H₂O₂ chamber 646, an air chamber 650, an evaporator 654, and a cell 656. The H₂O₂ chamber 646 is fluidly coupled to the evaporator 654 via an H₂O₂ conduit 658. The H₂O₂ conduit 658 transports H₂O₂ from the H₂O₂ chamber 646 to the evaporator 654. Similarly, the air chamber 650 is fluidly coupled to the evaporator 654 via an air conduit 662. The air conduit 662 transports air from the air chamber 650 to the evaporator 654. In other embodiments, the air conduit 662 may be coupled to external air, rather than the air chamber 650 such that the air conduit 662 draws in ambient air. In some embodiments, the H₂O₂ conduit 658 and the air conduit 662 are formed of the same material. In other embodiments, the H₂O₂ conduit 658 and the air conduit 662 may be formed of different materials. The H₂O₂ conduit 658 and/or the air conduit 662 may be formed of HDPE or stainless steel. In other embodiments, the H₂O₂ conduit 658 and the air conduit 662 may be formed of alternative materials.

The evaporator 654 is configured to receive the H₂O₂ and the air, which will be referred to as the combination, and mix and vaporize the combination. The evaporator 654 heats the combination to a vaporization temperature of 108 degrees Celsius. In other embodiments, the vaporization temperature may be below 108 degrees Celsius. In other embodiments, the vaporization temperature may be above 108 degrees Celsius. Once the evaporator 654 heats the combination, the H₂O₂ is vaporized, creating vaporized H₂O₂. The evaporator 654 is fluidly coupled to the cell 656 via an exit conduit 663. The exit conduit 663 may be formed of HDPE or stainless steel. In other embodiments, the exit conduit 663 may be formed of alternative materials. The combination including the vaporized H₂O₂ and the air travels through the exit conduit 663 and into the cell 656. The combination including the vaporized H₂O₂ and the air remains in the cell 656 until the combination is sprayed onto the preform 206 via a nozzle 664. The nozzle 664 is fluidly coupled to the cell 656. The cell 656 includes a cell heater 666 to heat the vaporized H₂O₂ if the H₂O₂ falls below the vaporization temperature. By heating the vaporized H₂O₂ to the vaporization temperature, residual H₂O₂ is prevented from remaining in the cell 656 and/or the nozzle 664. For example, the combination sprayed out from the nozzle 664 can form a cloud of vaporized H₂O₂ and air that interacts with surfaces of the preform 206 to sterilize the surfaces.

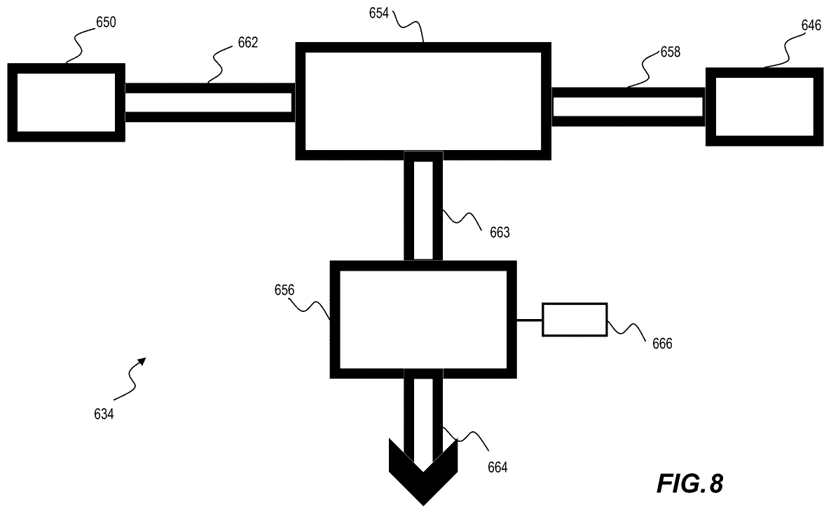

With reference to FIG. 9, the nozzle 664 includes a sensor monitoring system 668 for monitoring a temperature of the vaporized H₂O₂ when the H₂O₂ is within the nozzle 664 and alerting a user when the temperature falls below the vaporization temperature. The sensor monitoring system 668 includes a sensor 672, an alarm 676, and a controller 680. The sensor 672 is configured to measure a temperature of the vaporized H₂O₂ within the nozzle 664. The sensor 672 is in line with the nozzle 664 such that the sensor 672 interacts with the vaporized H₂O₂ to determine a temperature of the vaporized H₂O₂. In other embodiments, the sensor 672 may be positioned external from the nozzle 664. For example, the sensor 672 may be positioned on an external surface of the nozzle 664. In such embodiments, the sensor 672 may measure an external temperature of the nozzle 664 and the controller 680 may determine a temperature of the vaporized H₂O₂ within the nozzle 664 based on the signal from the sensor 672.

The alarm 676 is configured to activate based on a temperature of the vaporized H₂O₂ exceeding a predetermined range, as explained in greater detail below. The alarm 676 may be a visual indicator, an auditory indicator, or a similar indicator. Therefore, when the vaporized H₂O₂ is below the vaporization temperature, the alarm 676 alerts the user that vaporized H₂O₂ is below the vaporization temperature. The alarm 676 may include a plurality of alert outputs. For example, the alarm 676 may output a first output in response to the temperature of the vaporized H₂O₂ exceeding a first temperature range, and a second output in response to the temperature of the vaporized H₂O₂ exceeding a second temperature range. The alert outputs may define different volume levels, brightness, sound, light color, or the like. In other embodiments, the alarm 676 may output one alert output.

The controller 680 is electrically connected to the sensor 672, the cell heater 666 of the cell 656, and the alarm 676. In other embodiments, the controller 680 may be connected to other components in the VHP unit 634. The controller 680 receives a reading from the sensor 672 indicating the temperature of the vaporized H₂O₂ and turns on the cell heater 666, the alarm 676, and/or other components in the VHP unit 634 if the reading exceeds a particular range of temperatures (e.g., the first temperature range and the second temperature range described herein).

In some embodiments, the controller 680 may perform different functions for different ranges of temperatures. For example, the controller 680 may perform a first function for a desired temperature range, a second function for a safety temperature range, and third function for a shutdown temperature range. The ranges may be based on a percentage change from the vaporization temperature. For example, in the desired temperature range, the reading may be within one percent of the vaporization temperature. In other words, when the difference between the reading and the vaporization temperature is within one percent of the vaporization temperature, the reading is within the desired temperature range. The safety temperature range may be between one to two percent of the vaporization temperature. In other words, when the difference between the reading and the vaporization temperature is between one to two percent of the vaporization temperature, the reading is within the safety temperature range. The shutdown temperature range may be greater than two percent of the vaporization temperature. In other words, when the difference between the reading and the vaporization temperature is more than two percent of the vaporization temperature, the reading is within the shutdown temperature range. In other embodiments, the desired temperature range, the safety temperature range, and the shutdown temperature range may be defined by alternative percentages of the vaporization temperature. In other embodiments, the controller 680 may perform additional functions in connection with additional temperature ranges.

In other embodiments, the ranges of temperatures in response to which the controller 680 may perform various functions may be expressed as explicit temperature values. For example, in the desired temperature range, the reading may be plus or minus one unit of temperature (e.g., Celsius or Fahrenheit) from the vaporization temperature. In the safety temperature range, the reading may be between plus or minus one to plus or minus two from the vaporization temperature. In the shutdown temperature range, the reading may exceed plus or minus two from the vaporization temperature. In other embodiments, the desired temperature range, the safety temperature range, and the shutdown temperature range may be defined by alternative temperature values. In other embodiments, the controller 680 may include additional temperature ranges.

When the reading is within the desired temperature range, the controller 680 performs the first function. In some embodiments, the first function includes a waiting period during which subsequent readings from the sensor 672 are evaluated. In other words, when the reading is within the desired temperature range, the controller 680 does not alter the function of any component of the VHP unit 634.

When the reading is within the safety temperature range, the controller 680 performs the second function. In some embodiments, the second function includes activating the alarm 676 and the cell heater 666. The alarm 676 may output a safety temperature output indicating that the temperature of the vaporized H₂O₂ exceeds the desired temperature range and is within the safety temperature range. In other embodiments, the second function may include activating the alarm 676 but not activating the cell heater 666, or activating the cell heater 666 but not activating the alarm 676. In other embodiments, the second function may include altering a flow rate of the vaporized H₂O₂. In other embodiments, the second function may include an alternative function.

When the reading is within the shutdown temperature range, the controller 680 performs the third function. In some embodiments, the third function includes activating the alarm 676 and ceasing operation of the VHP unit 634. In other words, when the reading is within the shutdown temperature range, the controller 680 shuts down the VHP unit 634 and activates the alarm 676. The alarm 676 may output a shutdown temperature output. The shutdown temperature output may be different than the safety temperature output. For example, the shutdown temperature output may include a light of a first color (e.g., red) and the safety temperature output may include a light of a second color (e.g., yellow). In other embodiments, the third function may define an alternative function. In other embodiments, the controller 680 may include additional functions for additional temperature ranges. For example, the controller 680 may perform a fourth function when the reading is within the additional temperature range. The fourth function may define turning on the alarm 676, turning on the cell heater 666, altering the flow rate of the vaporized H₂O₂, and/or otherwise altering functionality of the VHP unit 634.

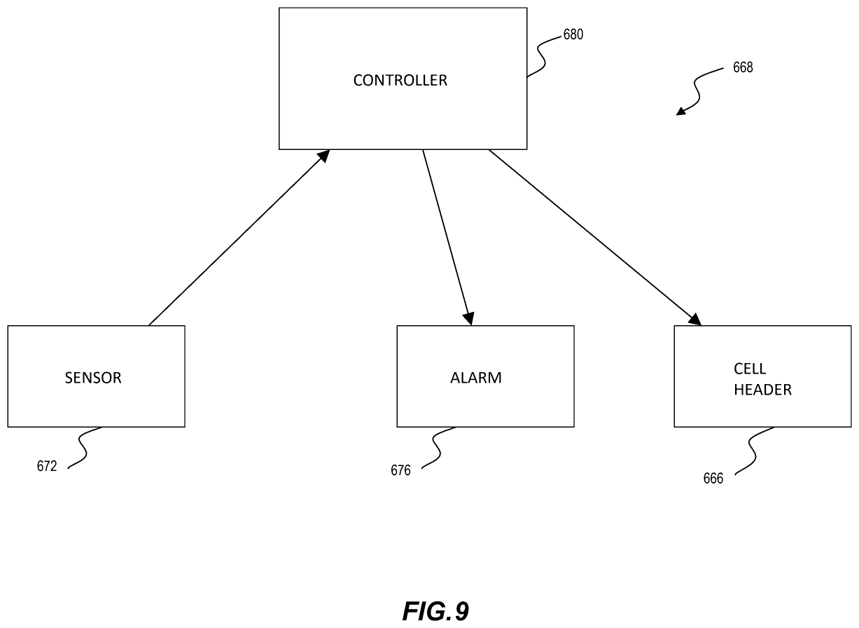

FIG. 10 shows a method 700 for detecting the temperature of the vaporized H₂O₂ within the nozzle 664. The sensor 672 detects the temperature of the vaporized H₂O₂ at the nozzle 664 (STEP 705). The sensor 672 provides a reading to the controller 680 corresponding to the detected temperature of the vaporized H₂O₂. The controller 680 determines whether the reading provided by the sensor 672 is in the desired temperature range, the safety temperature range, or the shutdown temperature range. If the reading is within the desired temperature range, the controller 680 awaits an additional reading from the sensor 672 (STEPS 710, 715). If the reading is within the safety temperature range, the controller 680 turns on the cell heater 666 and the alarm 676 (STEPS 720, 725). The cell heater 666 warms the vaporized H₂O₂ to the vaporized temperature. The alarm 676 alerts the user that the vaporized H₂O₂ is below the vaporized temperature. If the reading is within the shutdown temperature range, the controller 680 shuts down the VHP unit 634 and turns on the alarm 676 (STEPS 730, 735). The sensor 672 continues to measure the temperature of the vaporized H₂O₂ during operation of the VHP unit 634.

According to the H₂O₂ sterilization process described herein, after application of the H₂O₂ to the preform 206 (FIG. 1), the preform 206 undergoes a heating step at the heating unit 112. The heating step vaporizes the H₂O₂ and activates radicals to thereby sterilize the interior of the preform. However, several factors can affect the sterilization process and lead to inadequate sterilization. These factors can include inadequate application or coverage of the H₂O₂ on the interior surface of the preform, temperature discrepancies of the preform during application of the H₂O₂ which may require additional heating, and inconsistent or inadequate vaporization of the H₂O₂ during the heating step. The temperature discrepancies and inconsistent vaporization may result in residual H₂O₂ remaining within the preform. Residual H₂O₂ within the preform can decrease the effectiveness of the sterilization process and potentially compromise the quality of a final product or beverage filled into the bottle at the filling system 120.

The present disclosure, therefore, also includes an improved heating unit 112 that more consistently vaporizes the sterilizing agent. More particularly, the heating unit 112 ensures proper vaporization of the H₂O₂ without negatively affecting the preform 206.

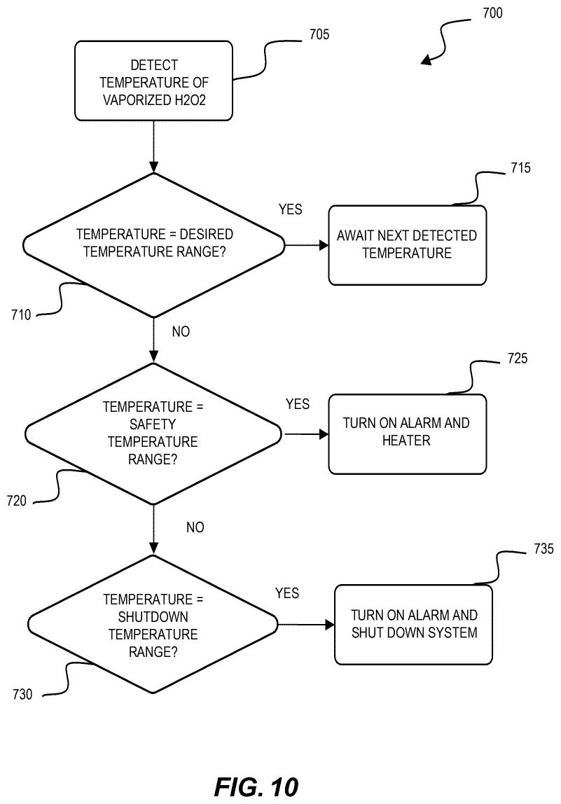

FIG. 11 is a schematic illustrating a hydrogen peroxide sterilization unit 110 and a heating unit 112 of the bottling line 100 according to an embodiment of the disclosure. In the embodiment of FIG. 11, the hydrogen peroxide sterilization unit 110 and the heating unit 112 can collectively be referred to as a sterilization system 111 for the preform 206. The system 111 is positioned upstream of the blow molding unit 116 (FIG. 1) and the filling system 120 (or filling station 120) to sterilize the preform 206 prior to molding and filling. The hydrogen peroxide sterilization unit 110 of FIG. 11 includes a vaporized hydrogen peroxide (VHP) unit 18 (or sterilizing agent applicator 18) followed by the heating unit 112 (or heating portion 112). The sterilizing agent applicator 18 is operable to apply H₂O₂ to an interior surface 20 of the preform 206. The sterilizing agent applicator 18 of the illustrated embodiment includes a rod 26 (or shaft 26 or conduit 26) that extends to a position within the preform 206 and proximate the interior surface 20. The rod 26 includes a nozzle 30 that sprays atomized droplets of the H₂O₂ onto the interior surface 20 of the preform 206. In the illustrated embodiment, the preform 206 is provided to the sterilizing agent applicator 18 at or near room temperature (e.g., at or near ambient temperature). However, in other embodiments, the preform 206 may be heated to a designated temperature just prior to application of the sterilizing agent (i.e., the H₂O₂ in the illustrated embodiment) as described herein. Following the application of the H₂O₂, the preform 206 is transported to the heating unit 112. The heating unit 112 includes an oven 34 through which the preform 206 is transported to heat the preform 206 and the H₂O₂. The oven 34 may be a convection oven, a radiant oven, or a combination thereof. The oven 34 may heat the preform 206 to a desired temperature at which the preform 206 may be blown into the bottle at the blow molding unit 116 (FIG. 1). The heat applied to the preform 206 by the oven 34 also may have the effect of vaporizing at least a portion of the H₂O₂ applied by the sterilizing agent applicator 18.

Under at least some conditions, the heat applied by the oven 34 to the preform 206 and the H₂O₂ may be insufficient to completely vaporize the H₂O₂, which can cause some residual H₂O₂ to remain on the interior surface 20 of the preform 206 and of the resultant bottle. With continued reference to FIG. 11, the heating unit 112 of the illustrated embodiment further includes a microwave heater 38 that compliments the oven 34 to effectively vaporize the H₂O₂. The microwave heater 38 (or microwave unit 38) includes a microwave generator 42 disposed upstream of the oven 34 and a reflector plate 46 disposed downstream of the oven 34. The microwave generator 42 directs microwaves towards the oven 34 to further heat the H₂O₂ on the interior surface 20 in parallel with the oven 34. The reflector plate 46 is operable to redirect microwaves that have passed through the oven 34 back towards the oven 34 and towards the preform 206 within the oven 34. In the illustrated embodiment, the reflector plate 46 is a metal plate disposed at or near an exit of the oven 34. In other embodiments, the reflector plate 46 may be formed of another material type capable of reflecting or re-directing microwaves. In other words, the reflector plate 46 redirects the microwaves back towards the oven 34 to decrease inadvertent excitation of peripheral objects in the environment other than the preform 206 being sterilized (e.g., objects or masses of the surrounding environment of the bottling line).

The microwaves emitted by the microwave heater 38 desirably affect or excite polar H₂O₂ molecules to generate heat while not affecting, or minimally affecting, non-polar molecules. The preform 206 is formed of a thermoplastic material, such as polyethylene terephthalate (i.e., PET), and may include additives that affect the stability and shelf life of a final product or bottle formed from the preform 206. The thermoplastic material may be non-polar or only slightly polar. Thus, as the preform 206 including the H₂O₂ passes through the heating unit 112, the oven 34 uniformly heats the preform 206 and H₂O₂ to begin the vaporization process of the H₂O₂. At the same time, microwaves from the microwave generator 42 primarily excite the H₂O₂ to increase the effectiveness of the vaporization process and reliably sterilize the preform 206. The combination of the oven 34 and the microwave heater 38 more effectively vaporizes the H₂O₂ and decreases the likelihood that residual H₂O₂ will remain within the preform 206 to potentially compromise the quality of a final bottled product or fail regulatory requirements. The oven 34 is optimized to heat the preform 206 for molding into the shape of a plastic bottle. The use of the microwave heater 38 to aid in the sterilization process allows for more effective vaporization of the H₂O₂ without significantly affecting heating of the preform 206 and therefore without affecting the molding process.

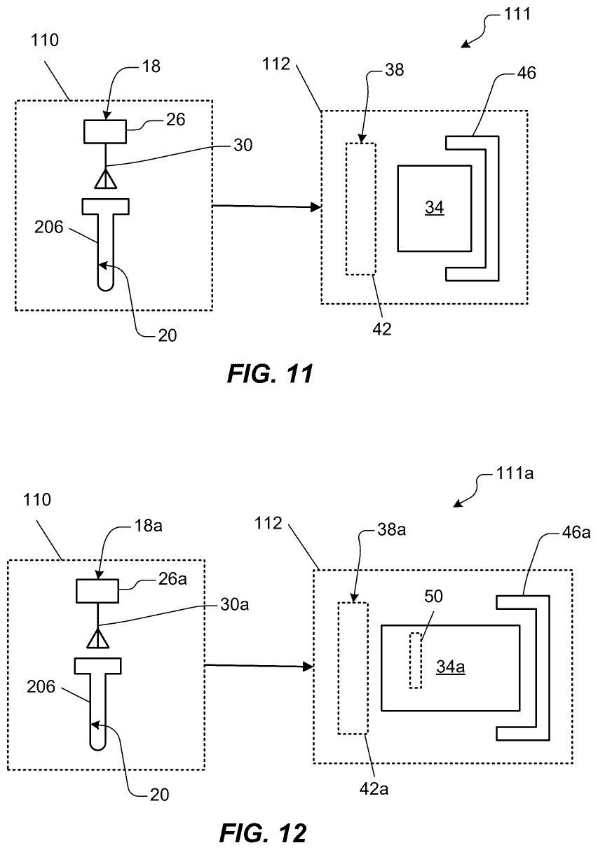

FIG. 12 illustrates a sterilization system 111a for sterilizing a preform 206 in accordance with another embodiment of the present disclosure with like parts having like reference numerals with the letter “a” appended thereon and the following differences explained below. Along with the reflector plate 46a, the microwave heater 38a further includes one or more wave guides 50 to direct microwaves towards the interior surface 20 of the preform 206. The wave guides 50, illustrated schematically in FIG. 2, may take the form of a rod that interacts with the microwaves to target the microwaves towards the interior of the preform 206. The wave guides 50 may alternatively form a cup disposed about an outer periphery of the preform 206 to target the microwaves towards the interior of the preform 206. In any shape or implementation, the wave guides 50 target or direct the microwaves toward the interior of the preform 206 and limit the extent to which the microwaves pass through the preform 206. As mentioned above, the PET preform 206 is not affected or only minimally affected by microwaves. However, the additives that improve the shelf life, among other things, of a final bottle formed from the preform 206 may be affected by the microwaves and lose effectiveness. Therefore, positioning the wave guides 50 to decrease the extent to which the microwaves pass through the preform 206 decreases the extent to which the final product may be affected by the microwave heater 38a.

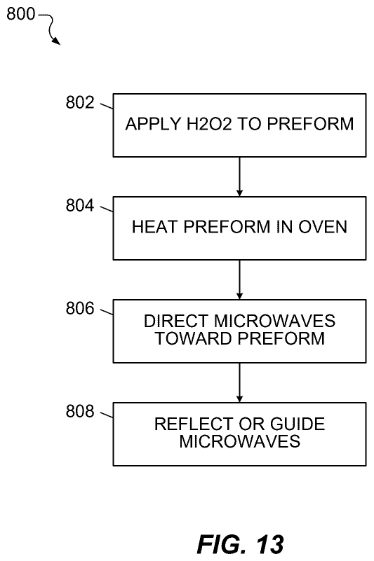

FIG. 13 is a flow chart illustrating a method 800 of sterilizing the preform 206. Step 802 of the method includes applying a sterilizing agent, such as H₂O₂, to the preform 206. Step 804 includes heating the preform 206 within an oven 34. Step 806 includes directing microwaves toward the preform 206 while the preform 206 is in the oven 34 to apply a secondary heating process to the sterilizing agent. The method 800 may further include a step 808 of directing the microwaves toward an interior of the preform 206 using a reflector plate 46 and/or a wave guide 50.

The foregoing description is merely illustrative in nature and does not limit the scope of the disclosure or its applications. The broad teachings of the disclosure may be implemented in many different ways. While the disclosure includes some particular examples, other modifications will become apparent upon a study of the drawings, the text of this specification, and the following claims. In the written description and the claims, one or more processes within any given method may be executed in a different order-or processes may be executed concurrently or in combination with each other-without altering the principles of this disclosure. Similarly, instructions stored in a non-transitory computer-readable medium may be executed in a different order-or concurrently-without altering the principles of this disclosure. Unless otherwise indicated, the numbering or other labeling of instructions or method steps is done for convenient reference and does not necessarily indicate a fixed sequencing or ordering.

It should also be noted that a plurality of hardware and software-based devices, as well as a plurality of different structural components may be utilized in various implementations. Aspects, features, and instances may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one instance, the electronic based aspects of the invention may be implemented in software (for example, stored on non-transitory computer-readable medium) executable by one or more processors. As a consequence, it should be noted that a plurality of hardware and software-based devices, as well as a plurality of different structural components may be utilized to implement the invention. For example, “control units” and “controllers” described in the specification can include one or more electronic processors, one or more memories including a non-transitory computer-readable medium, one or more input/output interfaces, and various connections (for example, a system bus) connecting the components.

Unless the context of their usage unambiguously indicates otherwise, the articles “a,” “an,” and “the” should not be interpreted to mean “only one.” Rather, these articles should be interpreted to mean “at least one” or “one or more.” Likewise, when the terms “the” or “said” are used to refer to a noun previously introduced by the indefinite article “a” or “an,” the terms “the” or “said” should similarly be interpreted to mean “at least one” or “one or more” unless the context of their usage unambiguously indicates otherwise.

It should also be understood that although certain drawings illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable connections or links.

Thus, in the claims, if an apparatus or system is claimed, for example, as including an electronic processor or other element configured in a certain manner, for example, to make multiple determinations, the claim or claim element should be interpreted as meaning one or more electronic processors (or other element) where any one of the one or more electronic processors (or other element) is configured as claimed, for example, to make some or all of the multiple determinations collectively. To reiterate, those electronic processors and processing may be distributed.

Spatial and functional relationships between elements—such as modules—are described using terms such as (but not limited to) “connected,” “engaged,” “interfaced,” and/or “coupled.” Unless explicitly described as being “direct,” relationships between elements may be direct or include intervening elements. The phrase “at least one of A, B, and C” should be construed to indicate a logical relationship (A OR B OR C), where OR is a non-exclusive logical OR, and should not be construed to mean “at least one of A, at least one of B, and at least one of C.” The term “set” does not necessarily exclude the empty set. For example, the term “set” may have zero elements. The term “subset” does not necessarily require a proper subset. For example, a “subset” of set A may be coextensive with set A, or include elements of set A. Furthermore, the term “subset” does not necessarily exclude the empty set.

In the figures, the directions of arrows generally demonstrate the flow of information—such as data or instructions. The direction of an arrow does not imply that information is not being transmitted in the reverse direction. For example, when information is sent from a first element to a second element, the arrow may point from the first element to the second element. However, the second element may send requests for data to the first element, and/or acknowledgements of receipt of information to the first element. Furthermore, while the figures illustrate a number of components and/or steps, any one or more of the components and/or steps may be omitted or duplicated, as suitable for the application and setting.

The term computer-readable medium does not encompass transitory electrical or electromagnetic signals or electromagnetic signals propagating through a medium-such as on an electromagnetic carrier wave. The term “computer-readable medium” is considered tangible and non-transitory. The functional blocks, flowchart elements, and message sequence charts described above serve as software specifications that can be translated into computer programs by the routine work of a skilled technician or programmer.

Although the disclosure has been described in detail with reference to preferred implementations, variations and modifications exist within the scope and spirit of one or more independent aspects of the disclosure as described.

### REPRESENTATIVE FEATURES

Representative features are set out in the following clauses, which stand alone or may be combined, in any combination, with one or more features disclosed in the text and/or drawings of the specification.

Clause 1. A method for manufacturing a bottle comprising: preheating a preform to an internal temperature with an airflow directed within a deduster unit; sterilizing the preheated preform with a hydrogen peroxide solution; and forming a bottle from the preheated preform using a blow molding process.

Clause 2. The method of clause 1, wherein the internal temperature is in a range of between about 45 degrees Celsius and between about 50 degrees Celsius.

Clause 3. The method of clause 1, wherein the airflow is preheated by a heating element.

Clause 4. The method of clause 3, wherein the airflow is ionized by an ionizer.

Clause 5. A system for manufacturing a bottle comprising: a deduster unit configured direct an airflow toward a preform to preheat the preform to an internal temperature; a hydrogen peroxide sterilization unit configured to sterilize the preheated preform to an internal temperature; and a blow molding unit configured to form a bottle from the preform.

Clause 6. The system of clause 5, wherein the internal temperature is in a range of between about 45 degrees Celsius and about 50 degrees Celsius.

Clause 7. The system of clause 5, wherein the deduster unit comprises a heating element configured to preheat the airflow.

Clause 8. The system of clause 7, wherein the deduster unit further comprises an ionizer configured to ionize the airflow.

Clause 9. A method for manufacturing a bottle comprising: preheating a preform to an internal temperature with a heating element; sterilizing the preheated preform with a hydrogen peroxide solution; and forming a bottle from the preheated preform using a blow molding process.

Clause 10. The method of clause 9, wherein the internal temperature is in a range of between about 45 degrees Celsius and between about 50 degrees Celsius.

Clause 11. The method of clause 9, wherein the heating element comprises a UV lamp.

Clause 12. The method of clause 9, wherein the heating element comprises an infrared heater.

Clause 13. A system for manufacturing a bottle comprising: a preparation unit configured to preheat a preform to an internal temperature; a hydrogen peroxide sterilization unit configured to sterilize the preheated preform to an internal temperature; and a blow molding unit configured to form a bottle from the preform.

Clause 14. The system of clause 13, wherein the internal temperature is in a range of between about 45 degrees Celsius and about 50 degrees Celsius.

Clause 15. The system of clause 13, wherein the preparation unit comprises a heating element configured to apply heat to the preform.

Clause 16. The system of clause 15, wherein the heating element comprises a UV lamp.

Clause 17. The system of clause 15, wherein the heating element comprises an infrared heater.

Clause 18. A sterilization system for a preform, the sterilization system comprising: a first conduit configured to carry H2O2; a second conduit configured to carry air; an evaporator fluidly coupled to the first conduit and the second conduit and configured to vaporize the H2O2 and the air, creating vaporized H2O2; a nozzle fluidly coupled to the evaporator and configured to deliver the vaporized H2O2 to the preform; and a heater configured to heat at least one of the H2O2 or the air prior to the at least one of the H2O2 or the air entering the evaporator.

Clause 19. The sterilization system of clause 18, wherein the first conduit includes the heater.

Clause 20. The sterilization system of clause 18, wherein the second conduit includes the heater.

Clause 21. The sterilization system of clause 18, wherein the second conduit includes a cooling device.

Clause 22. A sterilization system for a preform, the sterilization system comprising: a first conduit configured to carry H2O2; a second conduit configured to carry air; an first cell fluidly coupled to the first conduit and the second conduit and configured to heat the H2O2 to within a first temperature range; a second cell fluidly coupled to the first cell and configured to heat the H2O2 to within a second temperature range, the second temperature range being greater than the first temperature range; and a nozzle fluidly coupled to the second cell and configured to dispense the H2O2 from the second cell toward the preform.

Clause 23. The sterilization system of clause 22, wherein the H2O2 moves through the first cell at a first flow rate and the H2O2 moves through the second cell at a second flow rate, and wherein the first flow rate is less than the second flow rate.

Clause 24. The sterilization system of clause 22, wherein the first cell heats the H2O2 to a gas phase.

Clause 25. The sterilization system of clause 24, wherein the second cell heats the H2O2 from the gas phase to an activated gas phase.

Clause 26. The sterilization system of clause 25, wherein the first cell heats the H2O2 more than the second cell heats the H2O2.

Clause 27. A sterilization system for a preform, the sterilization system comprising: an evaporator configured to receive H2O2 and heat the H2O2, creating vaporized H2O2; a nozzle positioned downstream from the evaporator and fluidly coupled to the evaporator, the nozzle being configured to deliver the vaporized H2O2 to the preform; and a sensor positioned downstream from the evaporator and configured to detect a temperature of the vaporized H2O2 downstream from the evaporator.

Clause 28. The sterilization system of clause 27, further comprising a controller for controlling operation of the sterilization system based on a temperature reading of the sensor.

Clause 29. The sterilization system of clause 28, wherein the controller awaits additional temperature readings in response to the temperature reading being within a first predetermined range.

Clause 30. The sterilization system of clause 29, wherein the controller sounds an alarm and heats the vaporized H2O2 in response to the temperature reading being within a second predetermined range.

Clause 31. The sterilization system of clause 30, wherein the controller activates an alarm and stops operation of the evaporator in response to the temperature reading being within a third predetermined range.

Clause 32. A sterilization system for a preform, the sterilization system comprising: a sterilizing agent applicator configured to apply a sterilizing agent to an interior of the preform; an oven configured to heat the preform; and a microwave configured to at least partially vaporize the sterilizing agent.

Clause 33. A method of sterilizing a preform comprising: applying a sterilizing agent to an interior of the preform; heating the preform and the sterilizing agent in an oven; and directing microwaves toward the preform when the preform is in the oven to apply a secondary heating process to the sterilizing agent.

Clause 34. The method of clause 33, further comprising directing the microwaves toward the interior of the preform.

Clause 35. The method of clause 34, wherein directing the microwaves toward the interior of the preform includes directing the microwaves with a reflector plate positioned outside of the oven to contain the microwaves within the oven.

Clause 36. The method of clause 34, wherein directing the microwaves toward the interior of the preform includes directing the microwaves with a wave guide positioned within the oven.

Various features of the disclosure are set forth in the following claims.

What is claimed is:

1. A method for manufacturing a bottle comprising:
preheating a preform to an internal temperature with an airflow directed within a deduster unit;
sterilizing the preheated preform with a hydrogen peroxide solution; and
forming a bottle from the preheated preform using a blow molding process.

2. The method of claim 1, wherein the internal temperature is in a range of between about 45 degrees Celsius and between about 50 degrees Celsius.

3. The method of claim 1, wherein the airflow is preheated by a heating element.

4. The method of claim 3, wherein the airflow is ionized by an ionizer.

5. A system for manufacturing a bottle comprising:
a deduster unit configured direct an airflow toward a preform to preheat the preform to an internal temperature;
a hydrogen peroxide sterilization unit configured to sterilize the preheated preform to an internal temperature; and
a blow molding unit configured to form a bottle from the preform.

6. The system of claim 5, wherein the internal temperature is in a range of between about 45 degrees Celsius and about 50 degrees Celsius.

7. The system of claim 5, wherein the deduster unit comprises a heating element configured to preheat the airflow.

8. The system of claim 7, wherein the deduster unit further comprises an ionizer configured to ionize the airflow.
